(19) United States
(12) Patent Application Publication
(10) Pub. No.: US 2019/0076558 A1
(43) Pub. Date: Mar. 14, 2019

(54) GERMICIDAL UV-C TREATMENTS

(71) Applicants: Ke ZHANG-MISKE, Royal Oak, MI (US); Edgar J. DIETRICH, Redwood City, CA (US); Thomas J. CHAPATON, Sterling Heights, MI (US); Frances h. JAMES, Sunnyvale, CA (US); GM Global Technology Operations LLC, Detroit, MI (US)

(72) Inventors: KE ZHANG-MISKE, ROYAL OAK, MI (US); EDGAR J. DIETRICH, REDWOOD CITY, CA (US); THOMAS J. CHAPATON, STERLING HEIGHTS, MI (US); FRANCES H. JAMES, SUN NYVALE, CA (US)

(21) Appl. No.: 16/087,722

(22) PCT Filed: May 23, 2016
(86) PCT No.: PCT/US2016/033720

§ 371 Date: Sep. 24, 2018

(51) Int. Cl. A61L 2/10; A61L 2/26; B60N 2/00; B60Q 3/12; B60Q 11/00

(52) U.S. Cl. CPC A61L 2/10; A61L 2/26; B60N 3/002; B60Q 3/12; B60Q 11/005; B60N 2/002

(57) ABSTRACT

A number of variations may include placement of germicidal ultraviolet-C (UV-C) light treatments within vehicle interiors/exteriors to minimize material surface microbial growth and contamination, and sanitizes vehicle surfaces. A number of variations may include vehicles including UV-C light devices mounted in or on the vehicle to treat interiors/exteriors of the vehicle to minimize material surface microbial growth and contamination, and sanitize vehicle surfaces.

### TECHNICAL FIELD

The field to which the disclosure generally relates to includes germicidal UV-C treatment.

### BACKGROUND

Microorganisms may grow in and on vehicle interiors and exteriors.

SUMMARY OF ILLUSTRATIVE VARIATIONS

A number of variations may include placement of germicidal ultraviolet-C (UV-C) light treatments within vehicle interiors or exteriors to minimize material surface microbial growth and contamination, and sanitizes vehicle surfaces. A number of variations may include vehicles including UV-C light devices mounted in or on the vehicle to treat vehicle interiors/exteriors so as to minimize material surface microbial growth and contamination, and sanitize vehicle surfaces.

A number of variations may include a vehicle having a passenger compartment and one or more UV-C lights positioned in the passenger compartment constructed and arranged to sanitize one or more surfaces of the vehicle or an object in the passenger compartment.

A number of variations may include a method of providing a vehicle having a passenger compartment and one or more UV-C lights positioned in the passenger compartment constructed and arranged to sanitize one or more surfaces of the vehicle or an object in the passenger compartment, and exposing the one or more surfaces or the object to light from the UV-C.

Other illustrative variations within the scope of the invention will become apparent from the detailed description provided hereinafter. It should be understood that the detailed description and specific examples, while disclosing variations within the scope of the invention, are intended for purposes of illustration only and are not intended to limit the scope of the invention.

### BRIEF DESCRIPTION OF THE DRAWINGS

Select examples of variations within the scope of the invention will become more fully understood from the detailed description and the accompanying drawings, wherein:

FIG. 1 is a schematic illustration of a portion of a passenger compartment of a vehicle having a UV-C light mounted to the vehicle ceiling to shine light onto a variety of surfaces in the passenger compartment according to a number of variations.

FIG. 2 is a schematic illustration of a plurality of UV-C lights positioned in the passenger compartment of a vehicle according to a number of variations.

FIG. 3A is a schematic illustration of a vehicle steering wheel having a plurality of UV-C lights mounted in the steering wheel according to a number of variations.

FIG. 3B is a sectional view of a vehicle steering wheel having a plurality of UV-C lights mounted in the steering wheel according to a number of variations.

FIG. 3C illustrates a plurality of UV-C lights mounted in a vehicle steering wheel and including electrical leads extending through a spoke of the steering wheel, to a steering wheel hub to the steering wheel column according to a number of variations.

FIG. 4 is a schematic illustration of a vehicle stick shift having a plurality of UV-C lights mounted in the stick shift to shine light on a surface of the stick shift according to a number of variations.

FIG. 5 illustrates a cup holder in a passenger compartment of a vehicle, wherein the cup holder includes a UV-C light constructed and arranged to illuminate a transparent bottle containing a transparent liquid placed in the cup holder according to a number of variations.

FIG. 6A is a schematic illustration of a display or instrument panel in a passenger compartment of a vehicle, wherein a plurality of UV-C lights are constructed and arranged to sanitize the surface of the display/instrument panel according to a number of variations.

FIG. 6B illustrates an assembly including a display/instrument panel including an outer cover layer and a plurality of UV-C lights positioned between the display panel and the outer cover layer according to a number of variations.

FIG. 6C illustrates a sectional view of a display/instrument panel having a bevel or trim portion spaced a distance from the display/instrument panel in a plurality of UV-C lights positioned between the bevel/trim and the display/instrument panel according to a number of variations.

FIG. 7A illustrates the rear of a passenger compartment in a vehicle including a plurality of surfaces which may be sanitized by one or more UV-C lights permanently positioned in the passenger compartment according to a number of variations.

FIG. 7B illustrates a rear portion of a passenger compartment of a vehicle including a plurality of surfaces including a work station or meal tray which may be sanitized by a UV-C light permanently positioned in the passenger compartment according to a number of variations.

FIG. 8 is a schematic illustration of a closed vehicle passenger compartment of a vehicle including a UV-C light in the closed vehicle passenger compartment constructed and arranged to sanitize one or more surfaces of the closed compartment or an object positioned within the closed compartment according to a number of variations.

FIG. 9 is a schematic illustration of a portion of a closed vehicle passenger compartment including a glove box, auxiliary, and/or in-dash storage compartments, including a UV-C light constructed and arranged to sanitize surfaces of the glove compartment, auxiliary, and/or in-dash storage compartments according to a number of variations.

FIG. 10A-C are each schematic illustrations of a portion of a vehicle door having a door handle and at least one UV-C light position to shine light on the outer surface of the door handle or the under surface of the door handle according to a number of variations.

FIG. 11 is a schematic illustration of an electronic control system and a method of selectively timing the use of UV-C lights in a vehicle passenger compartment or on the outer surfaces of the vehicle, which may include, but is not limited to, limiting the use of the UV-C lights or locking out the use of the UV-C lights when there is a change that a human or a living being could be exposed to UV-C light, according to a number of variations.

### DETAILED DESCRIPTION OF ILLUSTRATIVE VARIATIONS

The following description of the variations is merely illustrative in nature and is in no way intended to limit the scope of the invention, its application, or uses.

A number of variations may include placement of germicidal ultraviolet-C (UV-C) light treatments within vehicle interiors/exteriors to minimize material surface microbial growth and contamination, and sanitizes vehicle surfaces. In the UV-C spectrum (100 nm to 280 nm), 250 to 280 nm is the most lethal wavelength range for microorganisms and is known as the germicidal spectrum, with 265 nm being the peak germicidal wavelength. The intense UV-C photon energy attacks the DNA of a living cell, penetrating the cell membrane, breaking the DNA structure of the microorganism and rendering it harmless by robbing it of the ability to reproduce. UV-C kills bacteria, viruses and the like, even if the organisms have developed immunity to other disinfection methods. A number of variations may include vehicles including UV-C light devices mounted in or on the vehicle to treat interiors/exteriors of the vehicle so as to minimize vehicle surface microbial growth and contamination, and sanitize vehicle surfaces. The term “mounted” as used here in means that the UV-C light devices are attached or secured in or on the vehicle by any one of a number of fasteners, mechanical features, adhesives, welding or the like that prevents the removal of the UV-C light device from the vehicle without removing a fastener, unlocking a mechanical feature or breaking an chemical or metallurgical bond to dismount the UV-C from the vehicle. In a number of variations the vehicle may be constructed and arranged to transport persons, animals and/or cargo. In a number of variations the vehicle may be constructed and arranged to transport persons, animals and/ or cargo. In a number of variations the vehicle may include an automobile (car), truck, bus, motor cycle, train, boat, trolley, cruise ship, military vehicle, autocycle, rickshaw, neighbor electric vehicle (golf cart), urban electric vehicle, personal electric vehicle, ocean vehicle, spacecraft, or aircraft.

A number of variations may include a vehicle having a passenger compartment and a UV-C light positioned in the passenger compartment constructed and strategically arranged to sanitize one or more surfaces or an object in the passenger compartment.

A number of variations may include method including providing a vehicle having a passenger compartment and a UV-C light strategically positioned in the passenger compartment constructed and arranged to sanitize one or more surfaces or an object in the passenger compartment, and exposing the one or more surfaces or one or more objects to light from the UV-C.

A variety of germicidal UV-C light emitting devices can be used. In a number of variations, the UV-C light emitting devices may be semiconductors such as light-emitting-diode (LED). In a number of variations the LEDs may be high power “Phlatlight” photonic lattice LEDs or multi-chip deep-UV LEDs.

The delivery of germicidal UV-C light may be controlled based on frequency, irradiation time, irradiation energy per surface area, wavelength, and light path distance from the source to the irradiated surface relative to the area to be treated. It may be controlled and monitored by an on-board computer or a remote system such as satellite communication system, for example OnStar, or a phone application.

The UV-C light device may be placed so that it is grounded via the vehicle chassis/body ground. This ground would also be considered the battery negative (−) ground. During service and maintenance operations, further steps to prevent accidental exposure to UV-C light include instructions to disconnect the battery negative (−) cable in order to disable the electrical system and prevent the UV-C light source from unintentionally activating.

UV absorbers, or hindered amine light stabilizers, may be optionally included in vehicle materials to prevent potential UV degradation of any surrounding polymer components that may be irradiated by the germicidal UV-C light.

In a number of variations, a germicidal UV-C semiconductor light source may be mounted onto a heat sink (passive heat exchanger) for adequate heat dissipation into surrounding medium.

In a number of variations, a germicidal UV-C semiconductor light source(s) can be operated through a control module and/or programmed via the vehicle's instrument panel. Lockout mechanisms that prevent the triggering of this system when the vehicle is occupied will also be necessary. An example of this system would be an algorithm that uses input from the seat sensors, an internal camera and infrared and/or motion sensors to detect when there are no human or pet occupants in the vehicle. The system may be activated at night to avoid additional environmental light interference. A timed dosage of germicidal UV-C irradiates the vehicle and is monitored by an on-board computer or remote system such as OnStar. OnStar or a similar cloud service notifies users that their vehicle has been disinfected.

Delivery systems may include direct or indirect illumination of the surfaces to be treated via UV-C LED.

Delivery systems may include indirect illumination of the surfaces to be treated using UV-C lights in combination with mirrors, fiber optics, filters, or light guides. Delivery systems may be integral to the surfaces being treated with light being transmitted through the surface (UV-C transparent) surface materials themselves.

In a number of variations the UV-C light may be placed in front and rear consoles, glove box, auxiliary, and/or in-dash storage compartments to enable the disinfection of the surfaces of personal items (e.g., personal electronics, makeup kits, food/liquid containers, toys, keys, pens/pencils, utensils, gloves).

In a number of variations the UV-C light may be placed in a sanitizing cup holder. Use in conjunction with UV-C transparent water bottle. Include locking mechanism to prevent vehicle occupant's accidental exposure.

In a number of variations the UV-C light may be placed in or on tray table surfaces sanitization. UV-C LEDs are activated to sanitize the surfaces when the tray table is closed. The light source may originate from UV-C transparent material as part of the seat back and/or the table surface assembly.

In a number of variations the UV-C light may be placed in or on frequently-touched surfaces so as to sanitize the same such as infotainment center, switches and rear-view/makeup mirrors could be illuminated from behind, the edges, or a bezel by UVC LEDs. A lockout device would be required to prevent accidental exposure to vehicle occupants.

The term “microorganisms” refers to bacteria, fungi, mold, mildew, algae, and viruses.

In a number of variations the UV-C emitting devices used in the vehicle may emit UV-C irradiation in the wavelength range 200 -280 nm, 250 -280 nm, 265nm, or any range within these ranges.

UV-C LEDs offer the advantages of a small size and minimal power consumption. They have a long life, are durable, robust, and are inexpensive.

The semiconductor light source may be contained in a surface mounted device (SMD) chip, star base chip, chip on board (COB), flip chip (FC), multi-chip, strip set, printed circuit board (PCB), photonic lattice chip, or transistor case package. Examples of surface mounted device chips are SMD-6868, -6565, -6060, -5730, -5630-5050, -3528, -3020, -3014, -2835. Examples of semiconductors with a round base are P5, P6, P7, P8, P10, P12; flip chips include LM131A, LH141A. A useful transistor case package is the TO-3/5/8/18/39/46/52/72

In a number of variations the semiconductor device may be one that employs aluminum gallium nitride (AlGaN) or aluminum indium gallium nitride (AlInGaN) multiple quantum wells (MQWs) over sapphire substrates or aluminum nitride (AlN) substrates. In a number of variations, the device may employ aluminum indium gallium nitride (AlInGaN) multiple quantum wells (MQWs) over a patented sapphire substrate (PSS) with backside roughening process as taught, for example in U.S. Pat. No. 9,112,103. In a number of variations the UV-C LED devices may be waterproof.

Referring now to FIGS. 1-9, a product may include a vehicle 10 having a passenger compartment 12. A passenger compartment as used herein means an area of the vehicle in which a driver or passenger may sit while the vehicle is being operated. The passenger compartment includes a variety of surfaces and compartments which a passenger may come in contact with while riding in the vehicle during normal operations and without having to disassemble portions of the vehicle to come in contact with the surface. The passenger compartment does not include heating, ventilation, and air conditioning (H-VAC) duct work.

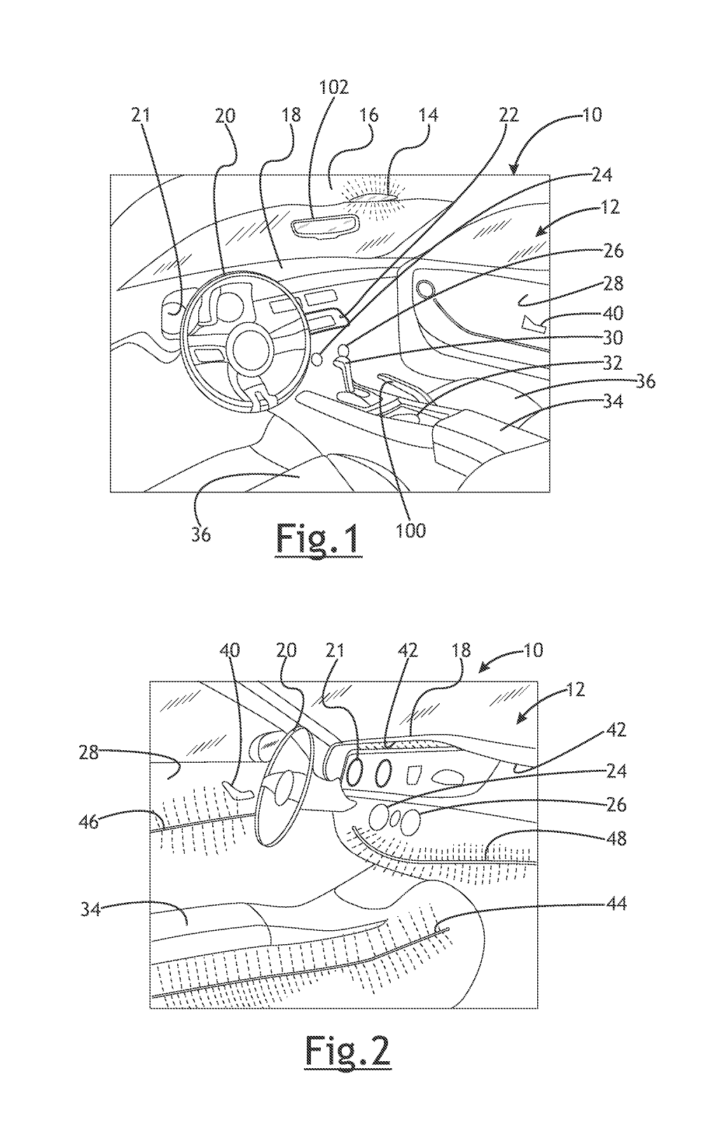

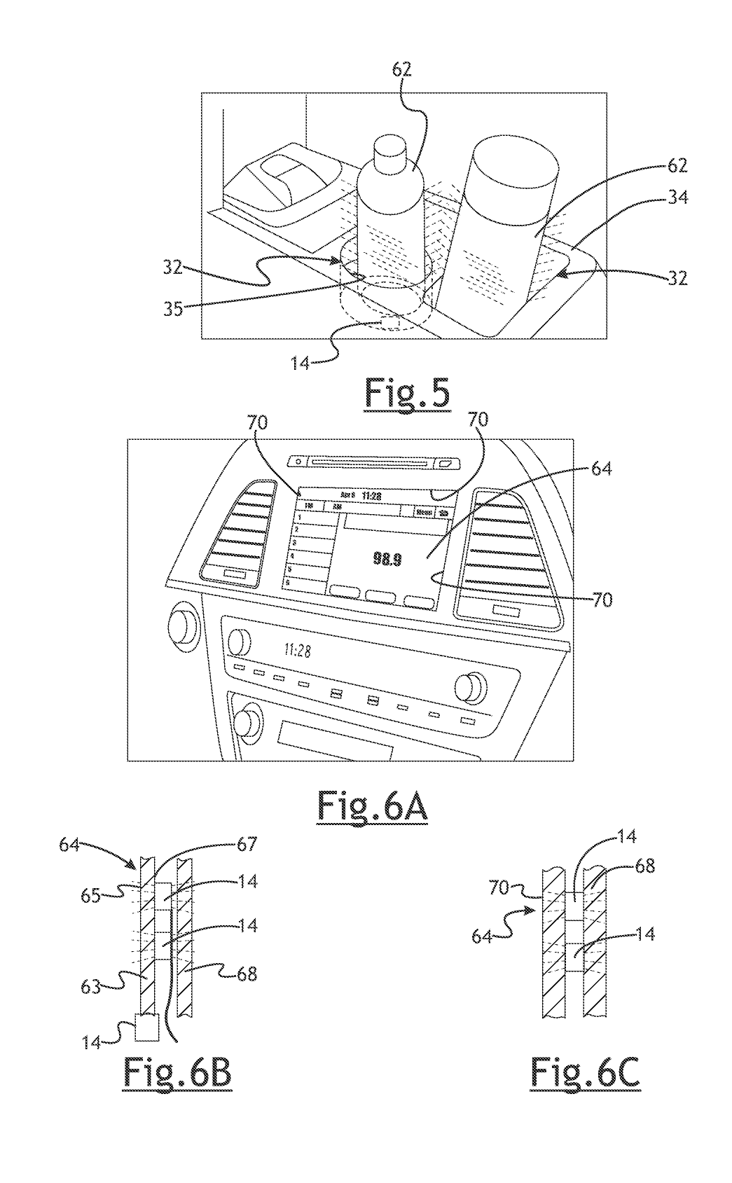

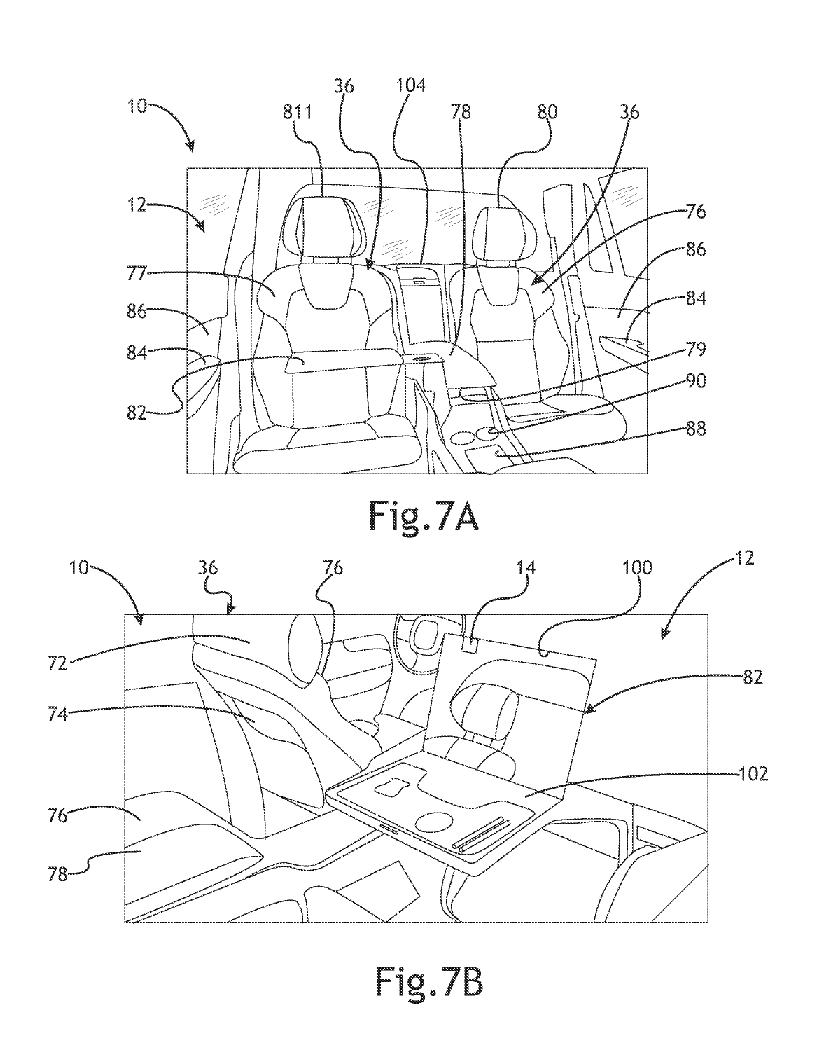

The UV-C light(s) 14 may be mounted in the passenger compartment 12 and constructed and arranged to shine light on and sanitize any of a number of vehicle surfaces, vehicle components or objects in the passenger compartment including, but not limited to, the dashboard 18, the steering wheel 20 (FIGS. 1-2, 3A), gages and displays 21 (FIGS. 1-2), surfaces of open compartments 22 (FIG. 1, 9), entertainment knobs and control surfaces 24 (FIGS 1-2), door surfaces 28 (FIGS. 1-2), stick shift 30 (FIGS. 1, 4), cup holder well 32 (FIGS. 1, 5), console surfaces 34 (FIGS. 1-2, 5), cup holder well walls 32 (FIG. 5), seat surfaces 36 (FIGS. 1, 7B) including head rest 72, 81, 80 (FIGS. 7A-B), and door handles 40 (FIGS. 1-2), emergency brake 100, rear view mirror 102 (FIG. 1), back panel 104 (FIG. 7A) . In one variation, one or more UV-C light(s) may be mounted to the ceiling 16 (FIG. 1) of the vehicle and constructed and arranged to shine light on any one of the surfaces in the passenger compartment 12.

Referring now to FIG. 2, in a number of variations a plurality of UV-C lights may be positioned in—a continuous pattern 46 in a door 28, in—a continuous pattern 44 along a portion of a central console 34, in a continuous pattern 48 along a lower portion of a dashboard 18, or in one or more continuation patterns on an under face 42 of the dashboard 18. In a a number of variations, the plurality of lights positioned in a continuous pattern may be in series spatially and/or electrically, or electrically in parallel. One or more continuous patterns of UV-C LED lights may be positioned to illuminate a number of surfaces, components or objects in the passenger compartment 12 of the vehicle.

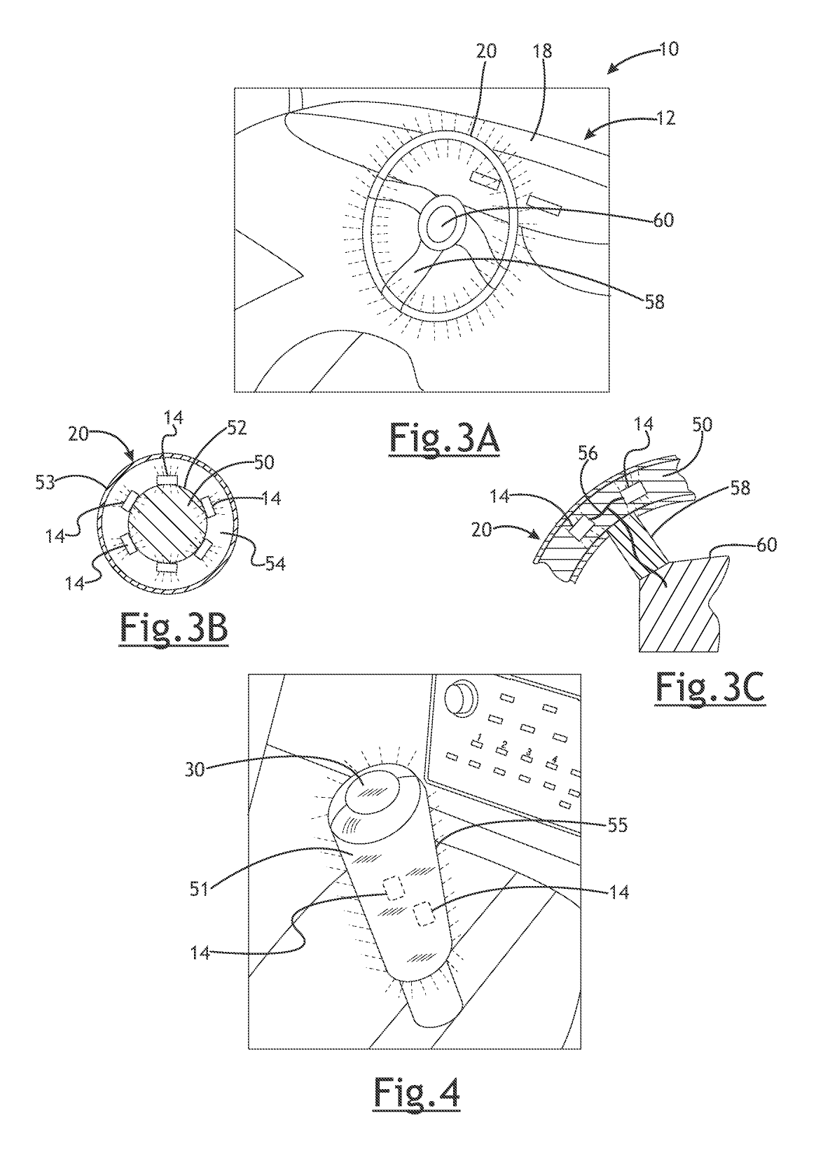

Referring now to FIG. 3A, in a number of variations, a plurality of LED lights may be mounted in a steering wheel 20 to illuminate transparent surfaces of the steering wheel which a passenger or driver may come in contact with.

Referring now to FIGS. 3B-C, in one variation, the steering wheel 20 may include a core portion 50 having an outer surface 52 on which a plurality of UV-C LED lights 14 may be mounted. A UV-C light transmissive layer 54 may be positioned over the plurality of UV-C LED lights 14 to allow transmission of light from the UV-C LED lights to the outer surface 53 of the steering wheel 20 which the driver or passenger may come in contact with.

Referring now to FIG. 3C, in a number of variations, a steering wheel including a plurality of UV-C LED lights mounted therein may also include one or more electrical leads 56 which may extend through a spoke 58 of the steering wheel and into a steering wheel hub 60 and wired from the hub 60 to an electrical control module.

Referring now to FIG. 4, in a number of variations a plurality of UV-C LED lights may be positioned in a stick shift 30. A UV-C light transmissive layer 51 may be provided over the plurality of UV-C LED lights and constructed and arranged to transmit UV-C light to an outer surface 55 of the stick shift 30 which may come in contact with a driver or passenger.

Referring now to FIG. 5, in a number of variations, a console 34 may include one or more cup holders 32 having cup holder well walls or surfaces 35 defining a cavity for receiving a cup or a bottle 62. A UV-C light may be mounted in the cup holder 32 and constructed and arranged to illuminate the contents of a bottle positioned in the cup holder 32.

Referring now to FIG. 6A, in a number of variations a display 64 may be provided and one or more UV-C LED lights may be constructed and arranged to illuminate the surfaces of the display or instrument panel 64 which a driver or passenger may come in contact with.

Referring now to FIG. 6B, in one variation, the display 64 may include an outer layer 63 having an outer surface 65 which a passenger or driver may come in contact with, and an opposite inner surface 67. An electronic display layer 68 may be provided and constructed and arranged to display information to the driver or passenger in the passenger compartment. A plurality of UV-C LED lights may be positioned between the outer layer 63 and the electronic display layer 68 and constructed and arranged to illuminate the outer surface 65 of the outer layer 63 to sanitize the same. In a number of variations, one or more UV-C lights may be positioned on the periphery of the outer layer 63 or within the outer layer to shine through the same. In one variation, the UV-C LED lights 14 may be attached to the inner surface 67 of the outer layer 63.

Referring now to FIG. 6C, in a number of variations, a bevel or trim 70 may be positioned along the periphery of the display 64. The bevel or trim 70 may be spaced a distance from the electronic layer 68 or the (optional) outer layer 63 (shown in FIG. 6B). A plurality of UV-C LED lights 14 may be constructed and arranged underneath the bevel or trim 70 so that UV-C light is shown onto the surfaces of the display 64, which a driver or passenger would come in contact with, to sanitize the same. In a number of variations, the structures illustrated in FIG. 6A-C may be used in electronic devices such as, but not limited to, cell phones, computer tablets, reading machines, gambling machines, bank machines and entertainment devices.

Referring now to FIG. 7A, the passenger compartment 12 may include a rear portion in which one or more rear seats 76, 77 are located. A rear console 78 may be provided between the rear seats 76, 77. Headrests 80, 81 may be provided for the rear seats 76, 77, respectively. One or more rear cup holders 90 and rear console compartment 88 may be provided in the rear console 78 or elsewhere. The rear console 78 may include a lift-up top 79. The rear console 78 may include a compartment constructed and arranged to store a meal (eating) or work station tray 82.

Referring now to FIG. 7A-B, in a number of variations, the meal or work station tray, or electronic device 82 may include a flip-up top 100 and a bottom 102. One or more UV-C LED lights may be mounted on the flip-up top 100 or the bottom 102 to illuminate surfaces of the meal or work station tray, or electronic device 82 which a passenger may come in contact with. The rear compartment 12 may include rear interior doors 86 having arm rests 84 which may be sanitized by a UV-C LED light positioned in the passenger compartment 12. The UV-C LED light positioned in the passenger compartment 12 may be positioned to illuminate any of the many surfaces which a passenger may come in contact with.

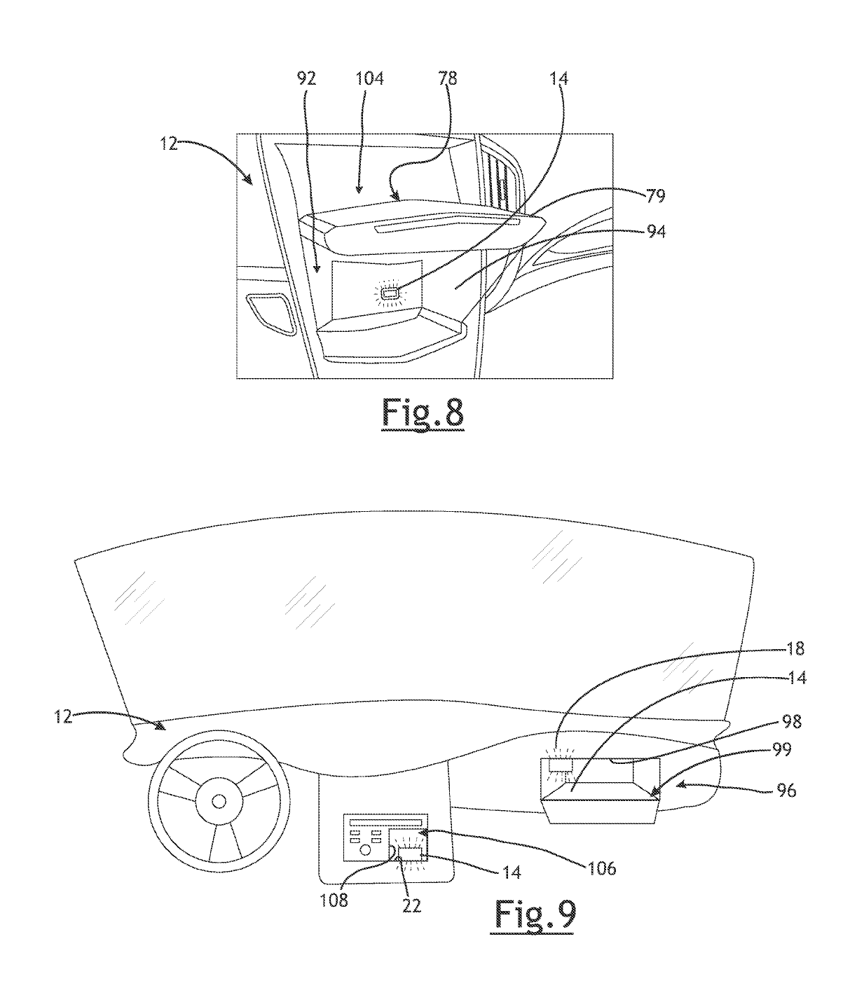

Referring now to FIG. 8, in a number of variations, a closed compartment 104, such as, but not limited to, a rear console 78, may be provided in the passenger compartment 12 and one or more UV-C LED lights 14 may be positioned in the closed compartment 104 to sanitize one or more surfaces 94 defining a cavity 92 in the closed compartment 104.

Referring now to FIG. 9, in a number of variations, a glove box, auxiliary, and/or in-dash storage compartments 96 may be provided including a plurality of walls 98 defining a cavity 99 to store any one of a number of objects. At least one UV-C LED light may be mounted in the glove box, auxiliary, and/or in-dash storage compartments 96 and positioned to sanitize the walls 98 thereof or any object contained within the glove box, auxiliary, and/or in-dash storage compartments 96. Similarly, other compartments 106, such as, but not limited to, CD holder compartments may be provided in the passenger compartment 12. One or more UV-C LED lights 14 may be provided and constructed and arranged to sanitize surfaces 108 defining the compartment 106 or objects contained in the compartment 106 according to a number of variations.

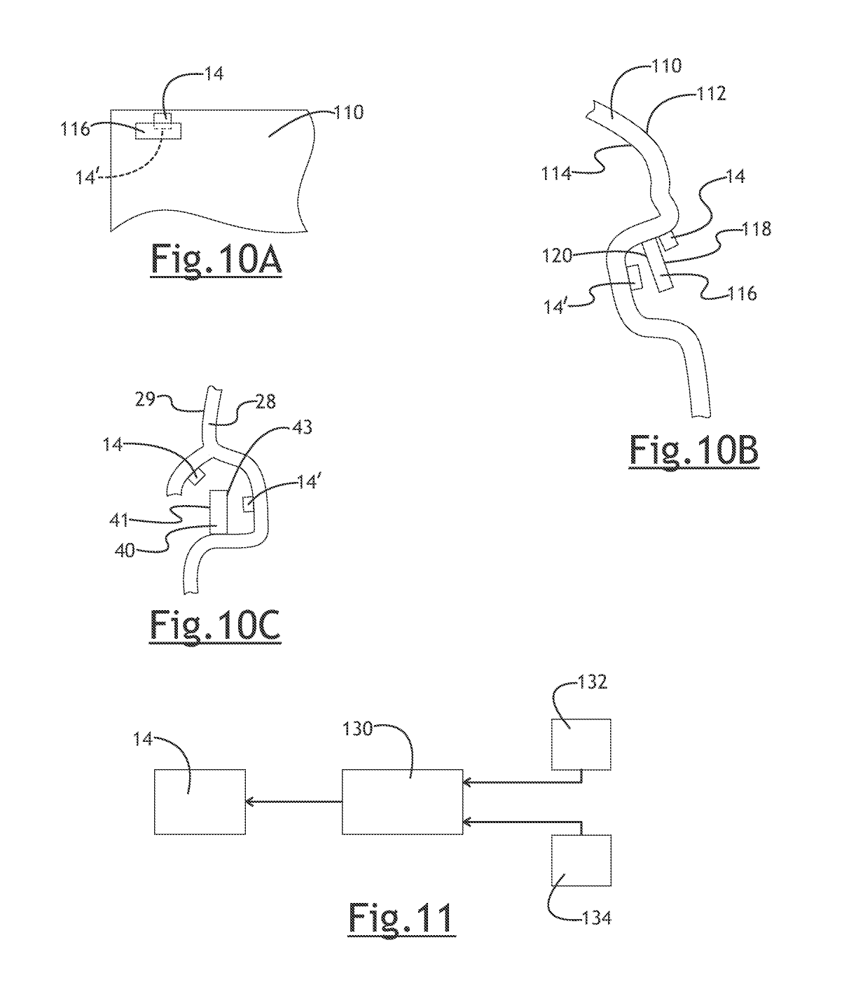

FIG. 10A-C are each schematic illustrations of a portion of a vehicle exterior door 110 having an exterior door handle 116 and at least one UV-C light 14, 14′ position to shine light on the outer surface of the exterior door handle 116 or the under surface of the exterior door handle 116 according to a number of variations. FIG. 10A is a partial of the exterior of an automotive vehicle door 110 having an exterior handle 116 with a UV-C light device 14 positioned to shine on an exterior surface of the exterior door handle 116 and another UV-C light device 141 shown in phantom lines positioned to shine on an inboard surface of the exterior door handle 116. FIG. 10B is a sectional view of an automotive vehicle exterior door 110 having an outer surface 112 and an inner surface 114, an exterior handle 116 pivotally attached to the door 110. The exterior door handle 116 may have an outer (outboard) surface 118 and a first UV-C light device 14 mounted in or to the door to shine on the outer surface 118 of the exterior door handle 116. The exterior door handle 116 may have an inner surface (inboard) 120 and a second UV-C light device 14′ may be mounted in or on the door 110 to shine on the inner surface 120 of the exterior door handle 116. FIG. 10C illustrates an interior door 28 of a vehicle having an inner (inboard) surface 29. An interior door handle 40 may be pivotally attached to the interior door 28 and a first UV-C light device 14 may be mounted in or on the interior door 28 to shine on an inner (inboard) surface 41 of the interior door handle 40 and a second UV-C light device 14′ may be mounted in or on the interior door 28 to shine on an outer (outboard) surface 43 of the interior door handle 40.

FIG. 11 is a schematic illustration of control system and a method of selectively timing the use of UV-C lights in passenger compartment on the outer surfaces of the vehicle, which may include, but is not limited to, limiting the use of the UV-C lights or locking out the use of the UV-C lights when there is a chance that a human or pet occupants could be exposed to UV-C light, according to a number of variations. A electronic control module 130 may be operatively connected or in communication with one of more sensors 132, 134 to detect the presence of a human or pet occupants being in the passenger compartment or near the exterior of the vehicle and may be utilized to selectively control the operation of one or more UV-C light devices 14 to avoid or limited exposure of humans or pet occupants to the UV-C light.

The above description of select variations within the scope of the invention is merely illustrative in nature and, thus, variations or variants thereof are not to be regarded as a departure from the spirit and scope of the invention.

1. A product comprising at least one germicidal ultraviolet-C (UV-C) light device mounted in or on a vehicle interiors or exteriors, or an electronic device, and constructed and arranged to shine light on one or more surface that a driver or passenger can come in contact with so as to minimize material surface microbial growth and contamination on the vehicle surfaces, or sanitize vehicle surfaces.

2. A product as set forth in claim 1 wherein the vehicle includes a passenger compartment and wherein the at least one UV-C light device is mounted in the passenger compartment and constructed and arranged to sanitize one or more surfaces or an object in the passenger compartment.

3. A product as set forth in claim 1 wherein the at least one UV-C light devices comprises a semiconductor device.

4. A product as set forth in claim 1 wherein the at least one UV-C light devices comprises a light-emitting-diode (LED).

5. A product as set forth in claim 1 wherein the at least one UV-C light devices comprises a high power phlatlight photonic lattice LED.

6. A product as set forth in claim 1 wherein the at least one UV-C light devices comprises a multi-chip deep-UV LED.

7. A product as set forth in claim 1 further comprising an electronic control system constructed and arranged to the at least one UV-C light device with respect to at least one of frequency, irradiation time, irradiation energy per surface area, or wavelength and light path distance from the source of the UV-C light to the one or more surfaces relative to the area to be treated.

8. A product as set forth in claim 8 wherein the electronic control system is at least one of an on-board computer or a remote system.

9. A product as set forth in claim 8 wherein the electronic control system is at least one of satellite communication system or a phone application.

10. A product as set forth in claim 1 wherein the vehicle comprises an automobile, truck, bus, motor cycle, train, boat, trolley, cruise ship, military vehicle, autocycle, rickshaw, neighbor electric vehicle (golf cart), urban electric vehicle, personal electric vehicle, ocean vehicle, spacecraft, or aircraft.

11. A product as set forth in claim 1 wherein the vehicle is constructed and arranged to transport persons, animals or cargo.

12. A method comprising providing a vehicle including one or more UV-C light devices mounted in or on a vehicle interiors or exteriors constructed and arranged to shine light on one or more surface that a driver or passenger can come in contact with so as to minimize material surface microbial growth and contamination on the vehicle surfaces, or sanitize vehicle surfaces, and exposing the vehicle surfaces to light from the one or more UV-C light devices.

13. A user interface component comprising:
a vehicle surface configured to come in to contact with a user; a semiconductor light source integrated within the surface; and a vehicle control circuit configured to deliver UV-C light via the semiconductor light source to the surface based on at least one of a predefined frequency, a predefined irradiation energy per a surface area, or a predefined light path distance.

14. A user interface as set forth in claim 13 wherein the surface a display.

15. A user interface as set forth in claim 13 wherein the surface an electronic display.

16. A user interface as set forth in claim 15 wherein the display has an outer surface and an electronic display layer, and wherein the semiconductor light source is integrated between the outer layer and the electronic display layer and constructed and arranged to illuminate on the outer surface.

17. A user interface as set forth in claim 13 wherein the control circuit is a vehicle processor configured to monitor the use of the semiconductor light source.

18. A user interface as set forth in claim 13 wherein the control circuit is a vehicle processor configured to selectively lock-out the use of the semiconductor light source.

19. A user interface as set forth in claim 13 wherein the vehicle control circuit configured to deliver UV-C light via the semiconductor light source to the surface based on at least one of a predefined frequency.

20. A user interface as set forth in claim 13 wherein the vehicle control circuit configured to deliver UV-C light via the semiconductor light source to the surface based on a predefined light path distance.
